(19) United States
(12) Patent Application Publication
(10) Pub. No.: US 2019/0142501 A1
(43) Pub. Date: May 16, 2019

(54) Surgical Instrument for Electrotomy and Tool for Same

(71) Applicant: Reger Medizintechnik GmbH, Villingendorf (DE)

(72) Inventor: Alexander Hetzel, Villingendorf (DE)

(73) Assignee: Reger Medizintechnik GmbH, Villingendorf (DE)

(21) Appl. No.: 16/093,374

(22) PCT Filed: May 30, 2017
(86) PCT No.: PCT/EP2017/000630

§ 371 Date: Oct. 12, 2018

(30) Foreign Application Priority Data
Jun. 15, 2016 (DE) 102016007232.6

(51) Int. Cl. A61B 18/14

(52) U.S. Cl. CPC A61B 18/148; A61B 2018/1422; A61B 2018/142; A61B 2218/007

(57) ABSTRACT

A surgical instrument for electrotomy and a tool for the same are disclosed. In an embodiment a tool includes a tubular main body having at its distal end an opening in which an active electrode is arranged, wherein the active electrode has a J-shaped electrode body having a short, bent first portion protruding through the opening of the main body and an elongate, tubular second portion extending coaxially in the main body, and wherein the first portion and the second portion are integral.

### CROSS-REFERENCE TO RELATED APPLICATIONS

This application is a national phase application under 35 USC 371 that claims the benefit of PCT/EP2017/000630, filed May 30, 2017, which claims the priority of German patent application 10 2016 007 232.6, filed Jun. 15, 2016, each of which is incorporated herein by reference.

### TECHNICAL FIELD

The invention relates to a surgical instrument for electrotomy and a tool for such a surgical instrument.

### BACKGROUND

The prior art discloses surgical instruments for electrotomy, which are used for endoscopic or arthroscopic operation methods, for example. Electrotomy should be understood to mean cutting or coagulating biological tissue, wherein small-area electrodes are used to produce a high current density at the transition spot of the current to the tissue. The electric power required to this end is selected in such a way that the tissue is heated to above 60° to above 100° Celsius in a very short time, the cell structure of the tissue is ruptured and the cytosol therein is vaporized. This technique is provided accordingly for arthroscopic electro surgery, namely for the treatment of tissue in body cavities or joints, and it is normally used in the presence of an expansion medium, which serves to inflate the body site to be treated, or of a saline fluid.

Electrotomy is also referred to as radiofrequency (RF) surgery, operated with alternating current at a frequency of approximately 300 kHz to 4 MHz provided to this end and can be applied in monopolar or bipolar fashion. In the case of the monopolar method, work is carried out using an active electrode at the tool and a neutral electrode, or else a return electrode, which is attached to the body part of the patient. The bipolar method is distinguished from the monopolar method by virtue of the neutral electrode being able to be arranged in the direct vicinity of the active electrode, directly on the tool. As a result, the path that the current has to take through the body is greatly shortened. To this end, use is made of different electrodes or electrode arrangements, which can be activated by way of different control elements.

To this end, German Patent DE 10 2011 105 404 B4 has disclosed a surgical instrument for electrotomy, having a rod-shaped electrode holder that is arranged at a handle. The electrode holder has a tubular main body that is made of an electrically insulating material and it carries an active electrode with a mushroom-shaped embodiment. Here, the active electrode protrudes through an opening of the distal end of the main body. Electrode and main body have a channel in the interior, said channel serving to suction away the tissue treated by the electrode.

A disadvantage here is that end regions are present in the main body and corner regions are present at transition portions from the electrode to the suction channel, at and in which regions tissue can accumulate. The tissue can only be removed poorly from the suction channel. The suction channel can become obstructed and the tool cannot be sterilized or only can be sterilized very poorly and, as a result thereof, can no longer be used anymore.

### SUMMARY

Embodiments of the invention provide an improved tool for a surgical instrument for electrotomy, by which tissue can be suctioned away more easily during an operation and which can be sterilized thoroughly.

Further embodiments of the invention provide a surgical instrument for electrotomy, which is easy to use and which can be produced in a cost-effective manner.

A tool for a surgical instrument for electrotomy according to embodiments has a tubular main body which has at its distal end an opening in which an active electrode is arranged. According to embodiments, the active electrode has a J-shaped or bent L-shaped electrode body having a short, bent first portion, which protrudes through the opening of the main body, and an elongate, tubular second portion, which extends coaxially in the main body. Here, the first portion and the second portion have an integral embodiment.

“J-shaped” or else “L-shaped” means an electrode body which has a bent part that is short in comparison with the overall length of the electrode body, and a shaft extending in an elongate manner adjoining therein. Here, proceeding from a longitudinal axis of this shaft, the bent portion points away from this shaft to the side in a manner angled through 90°. As a result of the first portion being bent, i.e., having a radius where edges were otherwise found, it is possible to virtually completely avoid an accumulation of tissue within the tool. There are no corners or edges within the suction channel, and so tissue that is detached by the electrode can readily be suctioned away quickly through the suction channel.

A particular advantage of this embodiment is that the electrode or the electrode body is constructed in integral fashion, i.e., constructed from a single continuously formed component, and it consists essentially of metal. Here, use can be made of the latest 3D printing technology, and so the electrode body can be manufactured in one piece. Complex milling or casting techniques are no longer necessary. Instead, the electrode body can be produced by means of additive production methods, such as 3D printing, for example. As a result, the tool can be constructed easily and it is also easy to sterilize as a result of the bent shape of the electrode.

The bent first portion can have one or more bend radii, which can be adapted according to the respective requirements. Thus, an inner first bend radius of the first portion may lie in a range from 0.3 mm to 3.0 mm and an outer second bend radius may lie in a range from 0.7 mm to 5.0 mm. As a result, it is possible to choose a bend of the first portion in such a way that suctioning that is as simple as possible can be achieved.

In a further embodiment, provision is made for the first portion of the electrode to be able to have a flange-like termination element with a protrusion that is radial in relation to the opening of the main body, said protrusion resting against an outer edge of the opening of the main body. The flange-like termination element forms an areal element that rests against the main body on the outside, completely covers the opening and moreover seals the latter. As a result of this, an electrode face is formed on the outer side of the main body, said electrode face being able to have a form and dimensions that can be chosen as desired. As a consequence, it is possible to realize different electrode forms, wherein the termination element may have a circular, oval or else rectangular form.

Further, the flange-like termination element can be a termination plate that has suction openings. Through these suction openings, tissue can easily enter the suction channel situated therebehind. These suction openings can have different dimensions, with it being possible to realize a single suction opening or else a plurality of smaller suction openings. If only a single suction opening is realized, it is possible to form a comparatively large opening which can receive relatively large tissue pieces and which comes into question for work at relatively large joints, such as for a hip, for example. If the termination plate has a plurality of smaller suction openings, these cannot be obstructed as quickly and they are well suited to work at relatively small joints, in which the detached tissue pieces are smaller, as a rule, such as for the shoulder region, for example.

In a further embodiment, provision can be made for the tubular main body to be of multipart design. Here, the main body can have a distal component and a proximal component, wherein an external diameter of the proximal component and an internal diameter of the distal component can be selected such that the proximal component is received in a detachable coaxial manner in the distal component. An overlap region of the distal component and proximal component forms a fastening portion for both components such that an assembled main body is formed.

Both components are substantially tubular and have an inner channel, which serves to suction away tissue. The distal component has a closed end side and, for the purposes of receiving the electrode body, has the opening of the main body in a laterally adjacent manner in the side wall of the distal component. Using this, tissue from sockets can be reached more easily and removed in a more targeted manner than when use is made of tools known from the prior art. The distal component can have an inner channel that is formed in the form of a blind hole. Alternatively, the channel can also follow the form of the electrode body and have a bend, which corresponds to the bend of the bent first portion and hence supports the portion.

The overlap of the two components can be designed in such a way that the distal end of the proximal component can partly rest on the inside against the end side of the distal component. When a connection between the inner channel of the proximal channel and the opening arises such that the electrode can be inserted, a distal end of the proximal channel is angled or has a corresponding recess. Here, a distal portion of the proximal component is embodied in such a way that the electrode body can be easily inserted. Since a wall of the proximal component can have a longer embodiment than an opposite wall, the electrode body can easily be inserted and also remains held in this position.

Furthermore, provision can be made according to embodiments for the proximal component, at its end forming the fastening portion, to have an external circumference taper by way of a step. The distal component can come to rest against the tapered external circumference, wherein an end of the distal component pointing to the step of the proximal component is spaced apart from the step. The step serves to form a fastening portion in which the proximal component can be crimped or welded on. This type of fastening is simple and produces a reliable and secure connection of the components.

This distance causes the formation of a fastening device such that the two components, in their form where they are plugged into one another, cannot detach from one another.

In various embodiments, an insulating layer is provided wherein the insulating layer rests around the proximal component and at least around part of the fastening portion. Here, the insulating layer can be a sleeve-shaped insulation, an insulating tube made of plastic or else shrink tubing. Here, the exposed external circumference taper of the proximal component and an end of the distal component form an abutment face for the insulating layer. This insulating layer or the shrink tubing can rest against both main body components, namely the distal component and the proximal component, in such a way that both are covered by the insulating layer in an overlapping manner. In the direction of proximal extent, the proximal component can also be covered completely by the insulating layer.

Various other embodiments provide for the distal component, at its end at which the fastening portion is present, to have an external circumference taper by way of a step and for the proximal component to have an internal circumference broadening by way of a step, wherein the proximal component rests against the tapered external circumference of the distal component. Here, the step of the distal component rests against the distal end of the proximal component and the step of the proximal component rests against the proximal end of the distal component such that both components overlap in a corresponding manner. The circumference taper/circumference broadening lie against one another here over the entire circumference. Insulation can be present over the entire external circumference or the lateral part of the proximal component and it can extend over the entire length thereof. If necessary, should the proximal component consist not of metal but of plastic, the insulation can also be dispensed with entirely.

Alternatively, embodiments can provide for the components of the main body to have latching elements that correspond to one another such that they can be connected to one another by latching in the fastening portion. However, as soon as the components are assembled, i.e., plugged into one another, they can be squeezed, crimped or else welded or adhesively bonded to one another so as to remain securely in the assembly arrangement.

The fastening options according to embodiments allow the two components of the main body to be connected to one another in such a way that the distal component is securely held and the tool is securely insulated.

Embodiments can further provide for the electrode body to extend at least so far into the main body—extending in the proximal direction of the tool—that it can reach up to the step of the proximal component, preferably reach further than an inner increment or a ring step. The electrode body can be partly received in the proximal component or it can extend in the interior of the latter over its entire length. The two essential portions of the electrode body can have different lengths. The bent first portion can be short in comparison with the elongate second portion and the second portion can have many times the length in comparison therewith and extend far into the proximal component. The second portion, however, can also vary in terms of length. For good suctioning and a good hold, the second portion extends beyond the fastening portion of the main body, far into the inner channel of the proximal component or up to the proximal end thereof. However, the second portion may also be very short, wherein it may have the same or only slightly longer dimensions than the first portion. In a further embodiment, the electrode body or the inner channel thereof tapers in the distal direction such that the channel widens in the proximal direction. This promotes good suctioning such that the obstruction can easily be detached should tissue be jammed or trapped in the distal region.

In order to be able to achieve good contacting of the electrode body with the proximal component, embodiments further provides for the electrode body to have a thickened external diameter at its proximal end, the lateral face of which rests against the inner side of the proximal component. As a result, a sliding contact is formed, which facilitates a power supply of the electrode body and hence the use as an active electrode. However, the active electrode can also be contacted directly—to this end, the electrode body can extend coaxially to a proximal end of the tool and be contacted there in a suitable manner. Further, use can also be made of other contacting methods; thus, the use of strip conductors, plug-in contacts, spring contacts, etc., is also possible in addition to sliding contact.

Further embodiments provide for the tool to be a bipolar tool and to be able to have a neutral electrode, which can be arranged in a sleeve-shaped coaxial manner in relation to the proximal component of the main body. The neutral electrode can be formed up to the end of the proximal component, although this is not necessary. It can also extend along the main body in the distal direction up to a certain point such that there is a certain distance between the neutral electrode and the active electrode. This reduces the risk of short circuits.

In order that the tool does not lead to short circuits or to damage healthy tissue if use is made of a neutral electrode, a further insulation in the form of a layer, coating, a shrunk-on plastics tube or else a pushed-on plastics sleeve can be applied over the neutral electrode.

In order to be able to use the tool in an instrument for electrotomy, embodiments provide for the active electrode, and hence the electrode body, to be manufactured from metal, preferably a stainless steel, particularly preferably a medical stainless steel. A 3D printing method can be used for producing the electrode body, wherein the form can be adapted as desired to a tool to be manufactured on an individual basis or to specific electrode forms. It is also possible to use other additive production methods. Further, the distal component of the main body can consist of an electrically insulating material, preferably a plastic or a ceramic, and the proximal component of the main body can consist of metal. What this can achieve is that the electrode body is supplied well with current and that the latter is insulated to the outside. Alternatively, provision can also be made for the proximal component likewise to consist of a plastic; then, it is possible to dispense with separate insulation. In this case, the electrode can be contacted at its proximal end with the electric contacts present in the handle, for which purpose it is possible to provide sliding contacts or plug-in contacts.

Further, provision can be made for the neutral electrode to be surrounded, in turn, by a sleeve-shaped insulation layer or an insulation tube. Consequently, an electrode arrangement made of an active and a neutral electrode is achieved at a distal end of the tool only. In exemplary dimensions, a tool has a length of 100 to 300 mm, wherein, in comparison therewith, the distal end portion has a length of 10 to 100 mm and the neutral electrode takes up a portion of 5 to 10 mm thereof. As a result, a particularly convenient tool is created.

Embodiments also provide a surgical instrument for electrotomy having a handle and a tool according to embodiments that is detachably connectable to the handle.

Hence, a particularly simple instrument is obtained, which facilitates reliable and fast suctioning of tissue.

In further embodiments, provision is made for the handle to have at its proximal end a connection device, wherein the connection device is an electrical connection of the electrode to a power supply and/or a tube-shaped connection to a suction apparatus. In particular, the handle has an ergonomic embodiment and different operating elements, by means of which, for example, the electrode can be energized and the tool can easily be interchanged. A suitable handle is described, inter alia, in DE 10 2011 016 585. Many different structural options are known for receiving and holding the tool.

The tool can be assembled or produced in a specific manner. Initially, the components are manufactured by known production methods, such as injection molding, milling technology or else 3D printing. The electrode body, in particular, can be manufactured directly in an integral fashion by means of 3D printing technology or by means of another additive manufacturing process. After providing the components, these are assembled in a certain sequence. Initially, the electrode body is inserted into the distal component. As a result of the latter being made of plastic and the connection element resting in planar fashion, the opening need not be sealed separately. Following this, the proximal component can be inserted into the distal component, with the front, tapered portion of the proximal component being guided between the wall of the distal component and the wall of the electrode body. If the end side of the proximal component comes to rest against the inner end side of the distal component, the insulation is applied over the proximal component and a region of the distal component from the proximal direction; by way of example, an insulating tube is pulled over or shrink tubing is pulled over and shrunk on. If use is made of a neutral electrode, the latter is plugged on and, finally, a final insulation is applied from the proximal side. Then, the tool is ready to use in this form.

Technical advantages, particularly when the active head is printed, arise by way of the production by means of 3D printing. Already existing systems and handles also can be retrofitted or simply used with the tool according to embodiments. By way of its simple construction, too, the tool contributes to providing an instrument that is easy to use.

### BRIEF DESCRIPTION OF THE DRAWINGS

Further embodiments and some of the advantages that are associated with these and further embodiments will become clear and more easily understandable from the following detailed description with reference to the attached figures. Articles or parts of same, which are substantially the same or similar, may be provided with the same reference signs. The figures are merely a schematic illustration of an embodiment of the invention.

FIG. 1 shows a side view of a surgical instrument according to an embodiment;

FIG. 2 shows a further side view of the surgical instrument, which has been rotated in relation to FIG. 1;

FIG. 3 shows a sectional illustration through a distal end portion A-A of a tool according to an embodiment; and

FIG. 4 shows a sectional illustration through a distal end portion of the tool according to a further embodiment.

### DETAILED DESCRIPTION OF ILLUSTRATIVE EMBODIMENTS

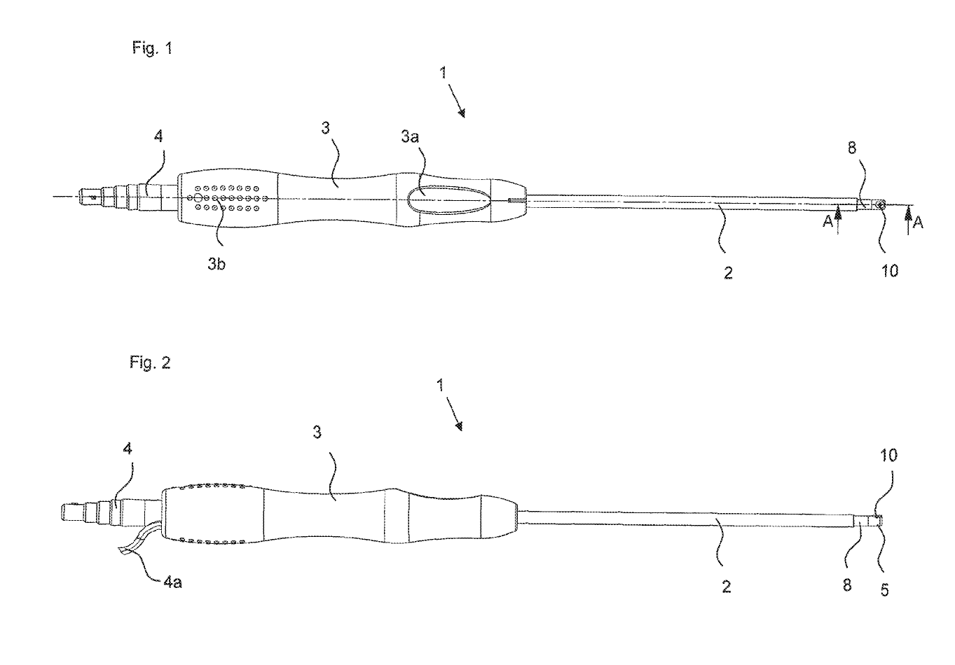

As shown in FIGS. 1 and 2, a surgical instrument 1 for electrotomy has a tool 2 which can be plugged onto a handle 3 in a detachable manner. The handle 3 is provided with an operating element 3a and pimples 3b for a non-slip hold of the handle 3. At its proximal end, the handle 3 has a connection device 4, which serves to connect a tube to a suction apparatus (not illustrated in the figures). Further, provision is made of an electric connection 4a on the connection device 4, which electrically connects the tool 2 to a power supply.

The tool 2 is rod-shaped and has, at its distal end region, an electrode 10, which is arranged laterally on the tool 2 at a main body 5. A further sleeve-shaped electrode 8 is arranged coaxially around the round main body 5. The electrode 10 mentioned first is used as an active electrode, whereas the sleeve-shaped electrode 8 is used as a neutral electrode.

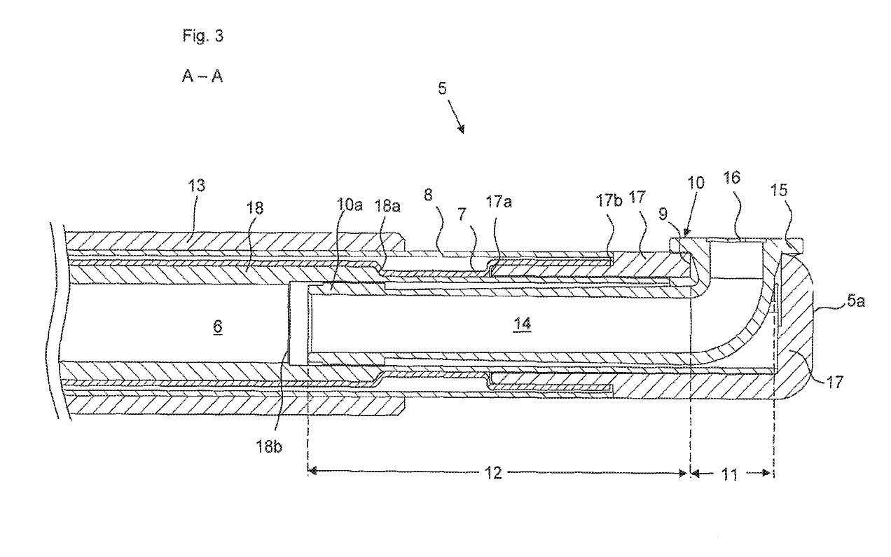

In accordance with the section A-A in FIG. 1, the distal end region is shown in more detail in FIG. 3, with the tool 2 substantially having a tube-shaped main body 5. The main body 5 is of multi-part design, it being constructed from a distal component 17 and a proximal component 18. The distal component 17 consists of a plastic, surrounds the electrode 10 and, at the same time, acts as insulation for the electrode 10. The proximal component 18 consists of a metal.

The proximal component 18 is tube-shaped and has a suction channel 6 and, via a step 18a, an external circumference taper. The distal component 17 is plugged onto the distal end of the proximal component 18 in such a way that it rests against the tapered external circumference of the proximal component 18. Here, an end of the distal component 17 pointing to the step 18a is at a distance from the step 18a. An external diameter of the proximal component 18 and an internal diameter of the distal component 17 are selected in such a way that the proximal component 18 is received in a detachable coaxial manner in the distal component 17 and both components overlap in a fastening portion.

An insulation 7 rests around the proximal component 18 and part of the fastening portion. The insulation can be shrink tubing, which is simply shrunk during the assembly of the tool. Here, the external circumference taper of the proximal component 18 and an end 17a of the distal component 17 form an abutment face for the insulation 7.

The insulation 7 extends in the distal direction up to a step 17b of the distal component 17, for which purpose the distal component 17 has a circumference taper at its proximal end region.

The electrode 10, which is arranged in the distal component, has an electrode body which is substantially received in the distal component 17. To this end, the distal component 17 has an opening 9, which is arranged not at an end side 5a of the main body 5 but at a side wall. This allows tissue from sockets to be reached more easily and removed in a more targeted manner.

The electrode body has an integral tube-shaped embodiment and can be produced very easily by 3D printing methods. It is substantially J-shaped and has two portions: a short, bent first portion 11, which protrudes through the opening 9 of the main body 5, and an elongate, tube-shaped second portion 12, which extends coaxially in the main body 5. Here, the two portions merge into one another because the electrode body has an integral embodiment; i.e., it is embodied as consisting of one piece. Since the electrode body is tubular, a channel 14 is formed in the interior of the electrode 10, said channel being connected to the suction channel 6.

The electrode body extends in a certain way in the main body 5, with it extending to behind the step 18a of the proximal component 18. At a proximal end 10a of the electrode body, the electrode 10 has a thickened external diameter, the lateral face of which rests against the inner side of the proximal component 18. Using this, the electrical connection to the proximal component 18, and hence to the power supply, is established. In order that the electrode body does not slip too far into the suction channel 6, a ring step 18b is formed on the outer side of the channel 6; however, said ring step is not formed incrementally, but conically. The ring step 18b is formed at the outer region of the active electrode 10. This ring step 18b can easily be embodied at the relevant region.

The first portion 11 of the electrode 10 has a flange-like termination element with a protrusion 15 that is radial in relation to the opening 9 of the main body 5, said protrusion resting against an outer edge of the opening 9 of the main body 5. Resting against the plastic of the distal component 17 acts like a seal between termination element and the main body 5.

The flange-like termination element is substantially a termination plate that has suction openings 16. These suction openings are directly connected to the channel 14 and, via the latter, to the suction channel 6 of the tool 2. Tissue which is detached by using the electrode 10 can be directly suctioned away through the electrode 10, with the tissue in the channel 14 simply being suctioned through the bent portion and not being able to get caught at any edge or corner.

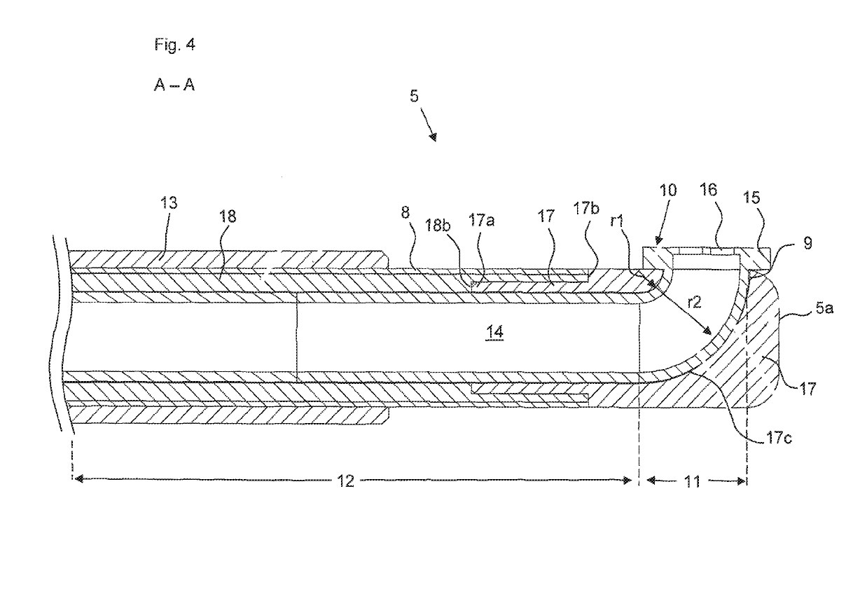

In FIG. 4, a further embodiment of the tool 2 exhibits an assembled arrangement of the individual components that has been modified in relation to FIG. 3.

At its end at which the fastening portion is present, the distal component 17 exhibits an external circumference taper by way of a step 17b. Corresponding thereto, the proximal component 18 has an internal circumference broadening by way of the step 18b, with the proximal component 18 resting against the tapered external circumference of the distal component 17. Here, the step 17b of the distal component 17 rests against the distal end of the proximal component 18 and the step 18b of the proximal component 18 rests against the proximal end 17a of the distal component 17, and so both components overlap in a corresponding manner.

In the variant of FIG. 4, the proximal component 18 consists not of metal but of a plastic. Therefore, a separate insulation is not necessary. Contacting the electrode 10 is effected at the proximal end thereof. To this end, the second portion 12 of the electrode 10 extends up to an electric connector, which engages with the electric connection device 4 of the handle 3 in an assembly arrangement. Use can be made of a sliding contact, a spring contact or else any other contact. The neutral electrode 8 rests around the entire length of the proximal component 18 as a sleeve. An insulating tube 13 is arranged coaxially to the neutral electrode 8 in order to embody the neutral electrode 8 in a ring-shaped manner and further insulate the shaft of the tool 2.

Further, the bent, first portion 11 of the electrode 10 is provided with different bend radii. An inner radius r1 of the first portion 11 lies between 0.3 mm and 3 mm; an outer radius r2 of the first portion 11 lies between 0.7 mm and 5 mm. In the region in which the first portion 11 of the electrode 10 is held, the distal component 17 is formed in such a way that the bent first portion 11 is completely surrounded. The inner channel of the distal component 17 corresponds in terms of form and dimensions to the form and dimensions of the sheath of the electrode body of the electrode 10. Hence, a bent inner surface 17c, which exactly maps the bend in the first portion 11 of the electrode 10, arises.

1-11. (canceled)

12. A tool for a surgical instrument for electrotomy, the tool comprising:
a tubular main body having at its distal end an opening in which an active electrode is arranged,
wherein the active electrode has a J-shaped electrode body having a short, bent first portion protruding through the opening of the main body and an elongate, tubular second portion extending coaxially in the main body, and
wherein the first portion and the second portion are integral.

13. The tool as claimed in claim 12, wherein the first portion of the electrode has a flange-like termination element with a protrusion that is radial in relation to the opening of the main body, and wherein the protrusion rests against an outer edge of the opening of the main body.

14. The tool as claimed in claim 13, wherein the flange-like termination element is a termination plate that has suction openings.

15. The tool as claimed in claim 12, wherein the main body comprises a distal component and a proximal component, wherein an external diameter of the proximal component and an internal diameter of the distal component are selected such that the proximal component is received in a detachable coaxial manner in the distal component, and wherein an overlap region of the distal component and proximal component forms a fastening portion.

16. The tool as claimed in claim 15, wherein the proximal component, at its end forming the fastening portion, has an external circumference taper and the distal component is able to rest against the external circumference taper, and wherein an end of the distal component pointing to the external circumference taper of the proximal component is spaced apart from the external circumference taper.

17. The tool as claimed in claim 16, wherein the external circumference taper is a step.

18. The tool as claimed in claim 16, wherein the distal component, at its end forming the fastening portion, has an external circumference taper and the proximal component correspondingly has an internal circumference broadening, and wherein the proximal component is able to rest against the external circumference taper of the distal component.

19. The tool as claimed in claim 18, wherein the external circumference taper of the distal component is a step, and wherein the internal circumference broadening of the proximal component is a step.

20. The tool as claimed in claim 16,
wherein an insulating layer is arranged around the proximal component and at least around part of the fastening portion, and
wherein the external circumference taper of the proximal component and/or an end of the distal component form an abutment face for the insulating layer.

21. The tool as claimed in claim 20, wherein the insulating layer is a sleeve-shaped insulation.

22. The tool as claimed in claim 16, wherein the electrode body extends at least so far into the main body that it reaches up to the external circumference taper of the proximal component and/or wherein the electrode body has a thickened external diameter at its proximal end, the lateral face of which abuts an inner side of the proximal component.

23. The tool as claimed in claim 12, wherein the tool is a bipolar tool having a neutral electrode, which is arranged in a sleeve-shaped coaxial manner in relation to a proximal component of the main body.

24. A surgical instrument for electrotomy comprising:
a handle; and
the tool according to claim 23,
wherein the tool is detachably connectable to the handle.

25. The instrument as claimed in claim 24, wherein the handle comprises, at its proximal end, a connection, and wherein the connection is an electrical connection of the electrode to a power supply and/or a tube-shaped connection to a suction apparatus.
